(19) United States
(12) Patent Application Publication
(10) Pub. No.: US 2026/0315582 A1
(43) Pub. Date: Oct. 15, 2026

(54) A VACCINE AGAINST LEPTOSPIRA BACTERIUM

(71) Applicant: INTERVET INC., Rahway, NJ (US)

(72) Inventor: Antonius Arnoldus Christiaan JACOBS, Kessel (NL)

(73) Assignee: INTERVET INC., Rahway, NJ (US)

(21) Appl. No.: 19/482,097

(22) PCT Filed: May 6, 2024
(86) PCT No.: PCT/EP2024/062453

§ 371 Date: Nov. 6, 2025

(30) Foreign Application Priority Data
May 12, 2023 (EP) 23173017.7

(51) Int. Cl. A61K 39/02; A61K 9/00; A61K 39/00; A61K 39/116; A61K 39/23; A61K 39/39; A61P 31/04

(52) U.S. Cl. CPC A61K 39/0225; A61K 9/0019; A61K 39/0241; A61K 39/116; A61K 39/23; A61K 39/39; A61P 31/04; A61K 2039/5254; A61K 2039/54; A61K 2039/545; A61K 2039/552; A61K 2039/55566; A61K 2039/70

(57) ABSTRACT

The invention pertains to a vaccine comprising a non-replicating Leptospira immunogen, for use in prophylactically treating a pig against an infection with a Leptospira bacterium, by injection of the first vaccine into the dermis of the pig.

The invention pertains to the prophylactic treatment of a pig against an infection with Leptospira bacterium by vaccination, and optionally against an infection with Erysipelothrix rhusiopathiae, and an infection with porcine parvo virus.

### BACKGROUND OF THE INVENTION

Pigs are prone to many infections and disorders arising from such infections, such as for example leptospirosis. This is a contagious disease of swine and many other animals (including humans) and is caused by infection with any one of a large group of

Leptospira bacteria (“lepto”). Leptospira are motile spirochetes, 6-12 microns long, and 0.1 micron in diameter. They usually are hooked on both ends and can be stained by Giemsa stain or, in tissue, by silver stains. In the laboratory they often are studied under darkfield microscopy. In laboratory media they are difficult to culture and grow very slowly (12-26 weeks). Many pathogenic strains can survive in the environment for long periods of time under moist conditions with a slightly alkaline pH. Most cannot withstand desiccation and are destroyed by common disinfectants.

Although there are generalities of leptospirosis in swine, details of infection with different serovars may vary. Swine are susceptible to many different serovars. Classification for Leptospira is based largely on DNA relatedness to known reference strains but universal agreement on taxonomy of the genus remains elusive. Under the most current classification, the family Leptospiraceae contains eight pathogenic species of which three are of most importance to swine: Leptospira interrogans (serovars pomona, icterohaemorrhagiae, canicola, and bratislava), Leptospira borgpetersenii (serovars sejroe and tarassovi) and Leptospira kirschneri (serovar grippotyphosa). Serovars pomona and bratislava are uniquely adapted to swine, others are maintained in other species but sometimes infect swine. Leptospira bratislava is reported to be the most common strain in swine, although the role of this serotype as a cause of disease is debated.

Control of leptospira is usually attempted by prevention of exposure, immunization through vaccination, and/or the use of antibiotics. Prevention of exposure is difficult to achieve because so many species can act as carriers of leptospires. These include infected swine, rodents (especially mice and rats) and many kinds of wildlife. Once leptospires are introduced into a favorable wet environment, they often persist there as a source of infection. Nonetheless, leptospirosis can be effectively controlled (perhaps even eradicated) from swine populations raised under confinement conditions using a combination of medication, vaccination, vector control, and providing a treated or non-contaminated drinking water source. Immunization through use of bacterins, widely practiced in breeding herds, usually will reduce the prevalence of infection and abortions. The bacterin must be appropriate for the serovar of Leptospira causing the disease. Many bacterins are thus multivalent.

Another example of a common swine disease is erysipelas, an infectious disease caused by Erysipelothrix rhusiopathiae (“ery”). This is one of the oldest recognized diseases that affect growing and adult swine. Up to 50% of pigs in intensive swine production areas are considered to be colonized with E rhusiopathiae. The organism commonly resides in the tonsillar tissue. Typically, healthy carriers shed the organism in their feces or oronasal secretions and are an important source of infection for other pigs. Infection is by ingestion of contaminated feed, water, or feces and through skin abrasions. When ingested, the organism can survive passage through the hostile environment of the stomach and intestines and may remain viable in the feces for several months. On farms where the organism is endemic, pigs are exposed naturally to E rhusiopathiae when they are young. Maternal-derived antibodies provide passive immunity and suppress clinical disease. Older pigs tend to develop protective active immunity as a result of exposure to the organism, which does not necessarily lead to clinical disease. Recovered pigs and chronically infected pigs may become carriers of E rhusiopathiae. Healthy swine also may be asymptomatic carriers.

Vaccination against E rhusiopathiae is very effective in controlling disease outbreaks on swine farms and should be encouraged. It may not be as effective in preventing chronic arthritis, however. Cessation of vaccination on some farms has been linked to disease outbreaks. Injectable bacterins and attenuated, live vaccines delivered via the water are available and provide extended duration of immunity. Optimal timing of vaccination may vary from farm to farm. When E rhusiopathiae is endemic in the production environment, vaccination should precede anticipated outbreaks. Susceptible pigs may be vaccinated before weaning, at weaning, or several weeks after weaning. Male and female swine selected for addition to the breeding herd should be vaccinated with a booster 3-5 weeks later. Thereafter, breeding stock should be vaccinated twice yearly with a follow up vaccination. Vaccination is however not always straight forward for arriving at protection. Vaccination failures may occur in some herds due to management stresses that compromise the immune system of vaccinated pigs. The use of live vaccines may also lead to clinical disease, particularly chronic erysipelas. Antigenic differences between serotypes in vaccines and serotypes circulating on farms could also result in incomplete immunity and disease outbreaks, but this is a rare event because there is thought to be good cross-protection among the major E rhusiopathiae strains infecting pigs.

Also, porcine parvovirus infection (PPV, or “parvo”) is a common pathogen of swine. It is an important cause of infectious infertility. PPV is a robust virus that multiplies normally in the intestine of the pig without causing clinical signs and is ubiquitous in pig populations world-wide. PPV is one of the organisms listed as responsible within the Stillbirths Mummification Embryonic Death and Infertility (SMEDI) syndrome. In larger herds it is almost certain to be present and is an infection one has to live with and manage, rather than try to eliminate. In smaller herds with a previously PPV positive pig, it may or may not have died out. Most viruses do not survive outside the host for any great length of time, PPV is unusual in that it can persist in the environment for many months and it is resistant to most disinfectants, and the most likely reason as to why is widespread and difficult to eliminate. PPV is most often transmitted either by mouth or through the snout, passing into the intestine where it multiplies and subsequently passed out in faeces. When a pig becomes infected for the first time there are no clinical signs. Boars may also infect a sow at the time of mating via their semen.

PPV cannot be eliminated from a herd so management and prevention of acute cases should be the aim. Routine vaccination of gilts and boars prior to entering the breeding herd plus yearly follow-up vaccinations of all pigs should be sufficient to ensure the herd is protected. It is known that if an infected breeding female has been vaccinated against PPV at some time in the past, then when exposure to PPV takes place, there is rapid re-stimulation of the immune system (within 5-7 days). This is sufficient to prevent disease and to stimulate a (semi-) permanent immunity.

PRRS virus was first reported in 1987 in North America and Central Europe. PRRS virus is a small, enveloped RNA virus. It contains a single-stranded, positive-sense, RNA genome with a size of approximately 15 kilobases. The genome contains nine open reading frames. The virus is a member of the genus Arterivirus, family Arteriviridae, order Nidovirales. The two prototype strains of PRRSV are the North American strain, VR-2332, and the European strain, the Lelystad virus (LV). The European and North American PRRSV strains cause similar clinical symptoms. In the early 2000s a highly pathogenic strain of the North American genotype emerged in China. This strain, HP-PRRSV, is more virulent than all other strains, and causes great losses in Asian countries worldwide. For any PRRS virus, subclinical infections are common, with clinical signs occurring only sporadically in a herd. Clinical signs include reproductive failure in sows such as abortions and giving birth to stillborn or mummified fetuses, and cyanosis of the ear and vulva. In neonatal pigs, the disease causes respiratory distress, with increased susceptibility to respiratory infections such as Glässer's disease.

Vaccine compositions against all of the above identified pathogens are commonly known.

### Object of the Invention

It is an object of the invention to provide a new method for convenient, safe and effective vaccination against one or more of the swine pathogens as mentioned here above.

### SUMMARY OF THE INVENTION

In order to meet the object of the invention, it has been found that a non-replicating Leptospira immunogen, can be used successfully in prophylactically treating a pig against an infection with a Leptospira bacterium, by injection of the (first) vaccine into the dermis of the pig. This way, safe and effective vaccination of a swine can take place in a convenient way. Although leptospira vaccines are available for swine, it is not a given that a new site of administration will lead to a safe and effective vaccine, even if the antigens are the same.

The World Health Organization (WHO) for example has published an e-learning course called “Vaccine Safety Basics” (https://apps.who.int/iris/handle/10665/340576), in which course on page 53 it is reported that “The route of administration is the path by which a vaccine (or drug) is brought into contact with the body. This is a critical factor for success of the immunization. A substance must be transported from the site of entry to the part of the body where its action is desired to take place. Using the body's transport mechanisms for this purpose, however, is not trivial.”

In this respect the California Department of Health Services' Immunization Branch has published guidelines for correct immunization (which can be found on the world-wide-web at https://www.cdc.gov/vaccines/pubs/pinkbook/vac-admin.html). With regard to the administration site it is stated on page 81, second full paragraph titled “Route and Site for Vaccination”, that “The recommended route and site for each vaccine are based on clinical trials, practical experience and theoretical considerations. There are five routes used to administer vaccines. Deviation from the recommended route may reduce vaccine efficacy or increase local adverse reactions.”

All in all, it is commonly known that vaccination at a particular site is not straightforward and requires experimentation to determine if safety and efficacy can be arrived at, and if so, if the level is adequate for commercial use.

Next to the vaccine as described here above, the invention pertains to a method of prophylactically treating a pig against an infection a Leptospira bacterium by injection of a first vaccine comprising a non-replicating Leptospira immunogen, into the dermis of the pig.

### Definitions

A vaccine is a pharmaceutical composition that is safe to administer to a subject animal, and is able to induce protective immunity in that animal against a pathogenic micro-organism, i.e. to induce a successful prophylactic treatment as defined here below.

Administration of a vaccine into the dermis means the vaccine is at least partly deposited in the dermis. The World health Organization in its Aug. 27, 2009 paper called “Intradermal Delivery of Vaccines; A review of the literature and the potential for development for use in low- and middle-income countries” indicates that “needle-less” vaccination does not necessarily mean “intradermal” vaccination (see Table 1, Page 3 of the review). Only when a needle-less device is “configured for intradermal vaccination”, then a vaccine may indeed be delivered into the dermis. Otherwise it may be delivered subcutaneous or intramuscularly. Several devices are commercially available for intradermal vaccination, for example the IDAL® vaccinator (MSD Animal Health), the Pulse 50 MicroDose (Pulse Needle Free Systems), or other devices as described in Vaccine, 2012 Jan. 11;30 (3): 523-38 (see in particular Table 1, page 525: “An overview of different devices for liquid and solid formulation administration”)

A combination of a first and second vaccine is a set of vaccines, wherein the first vaccine is individually distinct from the second vaccine, but wherein the two vaccines are used in conjunction for administration to the same subject in one concerted treatment.

Non-replicating immunogen of a pathogen is any substance or compound corresponding to the pathogen, other than the live replicating pathogen as a whole (either in wild type of attenuated form), against which pathogen an immunological response is to be elicited, such that the corresponding virulent pathogen or one or more of its virulence factors will be recognized by the host's immune system as a result of this immune response and are ultimately at least partly neutralized. Typical examples of non-replicating immunogens are killed whole pathogens (which term includes these pathogens in lysed form) and subunits of these pathogens such as capsid proteins, surface expressed molecules (for example recombinantly expressed proteins or lipopolysaccharides) and excreted molecules such as toxins.

A bacterin is a suspension of killed bacteria, either as whole cells, partly lysed or completely lysed (such as for example by homogenisation, French pressing, or a combination of two or more lysing methods).

A live attenuated pathogen is a viable, replication competent form of the pathogen having reduced virulence. The process of attenuation takes an infectious pathogen and alters it so that it becomes harmless or less virulent, typically by either multiple passages of the pathogen through cell systems or by genetically modifying the pathogen.

Prophylactic treatment against an infection with a pathogen is aiding in preventing, ameliorating or curing an infection with that pathogen or a disorder arising from that infection, resulting from a post treatment challenge with the pathogenic pathogen, in particular to reduce its load in the host after such challenge or to aid in preventing or ameliorating one or more clinical manifestations resulting from the post treatment infection with the pathogen.

A pig is an animal belonging to the family of Suidae, a family of artiodactyl mammals which are commonly called pigs, hogs or boars. Eight-teen extant species are currently recognized (or nineteen counting domestic pigs and wild boars separately), classified into between four and eight genera. Within this family, the genus Sus includes the domestic pig, Sus scrofa domesticus or Sus domesticus.

Associated separate administration of vaccines, also referred to as concurrent administration, is the administration of these vaccines separately, thus not mixed before administration, to the target animal, but within a time frame such that immunological interference is expected to occur, typically within 24 hours. A typical example of associated use is the simultaneous administration at separate application sites in the target animal, or separated in time by at most 1-24 hours.

Simultaneous administration of vaccines means the administration at exactly the same time or at least within a time frame of 1 hour, preferably within 55, 50, 45, 40, 35, 30, 25, 20, 19, 18, 17, 16, 15, 14, 13, 12, 11, 10, 9, 8, 7, 6, 5, 4, 3, 2 minutes or even a time frame of 1 minute, such as at the same time exactly

Single dose administration of a vaccine for use in prophylactically treatment means that in order to arrive at protective immunity, the vaccination does not need to be boosted with a second administration of the vaccine.

A prime and boost vaccination scheme means that in order to arrive at protective immunity, a first vaccination is boosted with a second administration of the vaccine. Typically the prime vaccination is boosted within 6 weeks, commonly within 5 or even 4 weeks from this first vaccination, and after the second (boost) vaccination adequate protective immunity is typically obtained.

### Further Embodiments of the Invention

In a first further embodiment of the vaccine according to the invention (i.e. the “first” vaccine, the non-replicating Leptospira immunogen is a Leptospira interrogans bacterin. Preferably, the vaccine additionally comprises a Leptospira kirschneri and/or Leptospira santarosai bacterin.

In another embodiment, the vaccine comprises an adjuvant. Despite the fact that it is commonly known that leptospira vaccines do not necessarily need an adjuvant to arrive at high levels of protection. The adjuvant, for example is an oil in water adjuvant. Typical adjuvants for use with the invention are oil-in-water adjuvants, such as emulsions of mineral oil in water, or mineral oil and vitamin-E-acetate in water. These adjuvants can be stabilised with emulsifiers such as polysorbate.

In an embodiment, the first vaccine for use according to the invention is injected by a jet stream of the vaccines using a needle-less device, wherein the jet stream penetrates the skin of the pig to reach the dermis.

In again another embodiment the first vaccine is for use in combination with a second vaccine, the second vaccine comprising a non-replicating immunogen of Erysipelothrix rhusiopathiae and a non-replicating immunogen of porcine parvo virus, in prophylactically treating a pig against an infection with the Leptospira bacterium, an infection with Erysipelothrix rhusiopathiae, and an infection with porcine parvo virus, by associated separate injection of the first vaccine and the second vaccine into the dermis of the pig at a first and a second injection site of that pig respectively. At typical intradermal vaccination volumes below 1 ml, typically around 0.2 ml, if the first and second injection sites are more than 1.5-2 cm's apart, the two vaccines will not mix underneath the skin and local reactions of each vaccine can be distinguished from the other. This means, that in line with the EMA Guideline on the requirements for combined vaccines and associations of immunological veterinary medicinal products (IVMPs), 18 Jul. 2013 (EMA/CVMP/IWP/594618/2010), which guideline defines “Separate sites” as application sites “sufficiently distant from each other to prevent the possibility of mixing of the products and to allow local reactions to each product to be distinguished from each other”, the injection sites can be regarded as “separate” in the sense of this embodiment of the present invention. With intradermal vaccination, any distance between the first and second injection site above 1.5, preferably above 2 cm, will thus lead to the same result of preventing negative interference regarding the safety and/or efficacy of the associated vaccination of the two vaccines, due to the lack of mixing of these two vaccines after administration.

In a further embodiment of this combination vaccination, the first and second injection sites are at most 4 cm apart from each other, preferably at most 3 cm apart from each other, even more preferably less than 2.5 cm such as 2 cm. It was found that this way pigs can be vaccinated more conveniently, in particular by using for example the IDAL® 3G Twin device (available from MSD Animal Health, Boxmeer, The Netherlands; distance between administration sites is 2.8 cm) having two barrels, holding separate vaccines, for associated intradermal injection of the two vaccines at the same time.

Further preferred is that the associated separate injection of the first vaccine and the second vaccine occur simultaneously.

In another preferred embodiment of the combination of a first and second vaccine for use according to the invention, the first and second vaccines are given in a prime and boost vaccination scheme, wherein both vaccines are injected at the prime vaccination, and both vaccines are injected at the boost vaccination.

In yet a further embodiment the non-replicating immunogen of Erysipelothrix rhusiopathiae is an Erysipelothrix rhusiopathiae bacterin, and the non-replicating immunogen of porcine parvo virus is an inactivated porcine parvo virus.

Advantageously, it was found that the combination of the first and the second vaccine for use according to any of the preceding claims, can also be used in conjunction with a third vaccine that is injected to the pig, the third vaccine comprising a live attenuated PRRS virus, and arrive at a safe and effective combination vaccination against all four pathogens, thus including protection against an infection with PRRS virus. Preferably, in the prophylactic treatment, the pig receives a prime vaccination with the first and second vaccine, and a boost vaccination with the first and second vaccine wherein at the boost vaccination (i.e simultaneous with or at least within 24 hours of this boost vaccination) the third vaccine is injected as a single dose vaccine. In a further embodiment the third vaccine (i.e. the PRRS vaccine) is mixed with the first vaccine (i.e. the Leptospira vaccine) for the boost vaccination.

The invention will now be explained in more detail using the following specific examples.

### EXAMPLES

Example 1 is a first trial of an Ery-Parvo-Lepto combination vaccine
Example 2 is a second trial of an Ery-Parvo-Lepto combination vaccine
Example 3 is a third trial of an Ery-Parvo-Lepto combination vaccine

### Example 1

Example 1 is a first trial of an Ery-Parvo-Lepto combination vaccine. For this the existing vaccine Porcilis® EPL was used (comprising E. rhusiopathiae, serotype 2 bacterin, inactivated porcine parvovirus, and various leptospira bacterins, in this case L. interrogans serogroup Icterohaemorrhagiae, L. interrogans serogroup Australis serovar Bratislava, L. kirschneri serogroup Grippotyphosa, L. interrogans serogroup Pomona serovar Pomona and lastly L. santarosai serogroup Tarassovi), which is known to be a safe and efficacious vaccine for use in swine, against ery, parvo and lepto infections by intramuscular vaccination. The antigens are all inactivated pathogens. Aim was to see whether the vaccine could also be applied intradermally and still meet predetermined safety and efficacy requirements. The device chosen for the intradermal delivery was the IDAL® 3G Twin device (the twin IDAL). Since the volume per administration is substantially less than what is recommended or Porcilis EPL, namely 0.2 ml instead of 2 ml, the amount of antigens of Porcilis EPL needed to be lowered (to 40 and 25%; see below) to be able and fitted into the smaller volume of the aqueous phase of the formulations.

Various different adjuvants were used in the test, namely the proprietary adjuvant Diluvac Forte (DF; MSD Animal Health, Boxmeer, The Netherlands), μDiluvac forte (μDF; same as Diluvac but with less surfactant and no mineral oil), and an experimental squalane, vitamin E-acetate and silica containing adjuvant (see i.a. WO 2021/048338), here denoted as SVEA. In the two Diluvac adjuvantia 40% of the antigen amount as present in Porcilis EPL could be formulated. In the squalene based adjuvant this was only 25%.

For the experiment healthy 12-week-old pigs, seronegative for PRRS and Ery, were used. The pigs were divided into 4 groups. The vaccine groups consisted of 10 animals and the control group of 5. Group 1 was vaccinated twice (in a prime-boost scheme) intradermally (ID) with EPL-ID-40%-DF with Porcilis PRRS administered non-mixed (as a separate vaccine) during the booster vaccination; Group 2 was also vaccinated twice (in a prime-boost scheme) ID, but in this group with EPL-ID-40%-μDF with Porcilis PRRS administered non-mixed during the booster vaccination. Group 3 was vaccinated twice ID with EPL-ID-25%-SVEA with Porcilis PRRS administered non-mixed during the booster vaccination. Group 4 was left unvaccinated. All vaccinations were administered intradermally (0.2 ml) in the neck (prime right side, boost left side) using the IDAL device and a 4-week interval between the vaccinations.

After vaccination, groups 1, 2 and 3 were observed for local injection site reactions. Three weeks after the last vaccination, groups 1 and 4 were challenged intradermally (0.1 ml), each of these groups in the right and left side of the chest with E rushiopathiae serotype 1 and serotype 2 challenge strains, respectively. Starting 2 days before challenge the animals were monitored for clinical signs and rectal temperature for 10 days. At each day of vaccination and at day of challenge blood was collected for Ery and Parvo serological examination.

Group 1 showed Ery antibody responses whereas the control group remained seronegative. None of the pigs in the groups tested induced an HI response against Parvo. The results are indicated here beneath in Table 1.

TABLE 1
Post vaccination and post-challenge data for EPL-ID

| | Local reactions | | | | | |
|---|---|---|---|---|---|---|
| | after boost | | Affected | Ph Eur req. | | |
| | avg/max | | pigs | for Ery | Ery sero | Parvo |
| Group | diameter | | st1/st2 | affected pigs | response | HI titre |
| 1 | 4.9/10 | cm | 20/30% | <10% | 64 | <4 |
| 2 | 6.7/12 | cm | — | — | — | — |
| 3 | 6.6/9 | cm | — | — | — | — |

| | | | | | |
|---|---|---|---|---|---|
| 4 | 0 | 100% | >80% | 0 | <4 |

None of the formulations was sufficiently safe (for this test set at 5/7 cm for avg/max local reactions) and the formulation tested for Ery efficacy was also insufficiently efficacious. In addition, this formulation did not induce a Parvo serological response. From the results it can be concluded that EPL is not feasible as vaccine for intradermal vaccination, both due to not meeting the safety and efficacy requirements.

### Example 2

Example 2 is a second trial of an Ery-Parvo-Lepto combination vaccine. In this trial, the ery parvo and lepto antigens were the same as in the previous trial. However, instead of formulating all antigens in one unitary vaccine, they were divided over two separate vaccines, namely an EP and an L vaccine. These vaccines were administered simultaneously as separate vaccines into the dermis of pigs using the IDAL device.

For the experiments three different adjuvants were used, the proprietary Emunade® and X-Solve® adjuvants (both oil-in-water adjuvants available from MSD Animal Health, Boxmeer, The Netherlands) and the above identified SVEA adjuvant.

Twenty healthy 18-week-old pigs, not vaccinated against any of the vaccine components, were used. The pigs were divided into 4 groups of 5 animals each. Groups 1, 2 and 3 were vaccinated twice intradermally with a 4-week interval (using the IDAL device) with EP-ID and L-ID (opposite side of the neck), using a vaccine containing 50% antigen content compared to Porcilis EPL (group 3) or 12.5% antigen content compared to Porcilis EPL (groups 1 and 2). The adjuvant Emunade was used in Group1, X-Solve in Group 2 and SVEA in Group 3. Group 4 was vaccinated with Porcilis EPL intramuscularly as a reference vaccination.

After vaccination, all groups were observed for local injection site reactions and serological responses to Ery, Parvo and against 3 of the 5 Lepto antigens i.e. Ictero, Pomona and Tarassovi antigens were determined.

The results are summarized in the Tables 2 and 3 below (responses are in relative units when no absolute units are given). Note that the parvo titres due to vaccination could not be determined since the animals underwent a field infection, resulting in high titres for all animals.

TABLE 2
Post vaccination and post-challenge data for EP-ID

| | Local reactions after boost | | | |
|---|---|---|---|---|
| Group | avg/max diameter | | Ery sero response | Parvo HI titre |
| 1 | 2.1/5.0 | cm | 79 | >10 log₂ |
| 2 | 2.6/3.0 | cm | 112 | >10 log₂ |
| 3 | 4.5/7.0 | cm | 91 | >10 log₂ |
| 4 | 0/0 | cm | 86 | >10 log₂ |

TABLE 3
Post vaccination and post-challenge data for L-ID

| | Local reactions after boost | | | | |
|---|---|---|---|---|---|
| Group | avg/max diameter | | L-ictero | L-pomona | L-tarassovi |
| 1 | 2.9/6.0 | cm | 3.8 | 5.4 | 2.0 |
| 2 | 1.7/3.0 | cm | 4.7 | 6.4 | 4.4 |
| 3 | 5.3/7.0 | cm | nd | nd | nd |
| 4 | 0/0 | cm | 6.2 | 6.7 | 5.2 |

For each adjuvant the site reactions were found acceptable and thus the vaccination was deemed safe. However, better results were obtained with the oil-in-water adjuvants, whereas the best results were obtained with the X-solve adjuvant, both regarding safety and efficacy. The latter is a dispersion of oil in water, which unlike Emunade, comprises no aluminium hydroxide.

The Ery antibody responses were comparable for all formulations and at the same level or higher when compared to Porcilis EPL. Parvo vaccination titres typically range between 2 and 10 log₂ whereas after a field infection, titres>10 log₂ may be found. From the results it is clear that a field infection had occurred during the study and thus no conclusions with regard to vaccination titres are possible. The Lepto antibody responses (Ictero, Pomona and Tarassovi) induced by the different formulations tended to be lower when compared to Porcilis EPL, but still deemed effective (as is known form the use of Porcilis EPL, a positive lepto titer of any level typically equals effective protection).

### Example 3

Example 3 is a third trial of an Ery-Parvo-Lepto combination vaccine, aiming at establishing the parvo response of the best vaccine of Example 2, namely the combination vaccine of Group 2, based on the oil-in-water adjuvant X-Solve. Also, the response against PRRS when vaccinating with a live attenuated PRRS vaccine when boosting the EPL prime vaccination was assessed.

Thirty-five healthy 12-week-old pigs weighing not less than 20 kg and PRRS negative and seronegative for Ery and Parvo were used. The pigs were divided into 4 groups (10 in each vaccine group and 5 in the control group). Group 1 was vaccinated twice ID (4-week interval) with EP-ID vaccine of Group 2 of Example 2 (right side) in associated non-mixed use with L-ID (of Group 2 of Example 2 (left side). In addition, during the booster vaccination Porcilis PRRS was administered non-mixed intradermally (left side) together with L-ID using Twin IDAL. Group 2 was vaccinated in the same way as group 1, except for the associated use with Porcilis PRRS. Group 3 was vaccinated identical to group 2 except that the EP-ID and L-ID vaccines contained only 25% of the antigen content (thus about 3% when compared with the antigen content of Porcilis EPL). Group 4 was left unvaccinated as negative control. All vaccinations were administered intradermally (0.2 ml) in the neck using IDAL or Twin IDAL. After vaccination, groups 1 and 2 were observed for local injection site reactions. At each day of vaccination and at day of challenge blood was collected for Parvo and PRRS serological examination.

At the start of the study, all pigs were seronegative for Ery, Parvo, Lepto and PRRS. The results are indicated here beneath in Table 4 (local reactions indicated corresponding to Tables 2 and 3)

TABLE 4
Post vaccination and post-challenge data for EP-ID plus L-ID

| | Local | | Local | | Parvo | PRRS |
|---|---|---|---|---|---|---|
| | reactions | | reactions | | titre | titre |
| Group | EP-ID | | L-ID | | 52 dpv | 52 dpv |
| 1 | 3.7/6.0 | cm | 3.8/5.0 | cm | 6.1 | 2.0 |
| 2 | 3.6/5.0 | cm | 3.0/4.0 | cm | 6.4 | 0 |
| 3 | nd | | nd | | 4.0 | 0 |
| 4 | 0/0 | cm | 0/0 | cm | <3 | 0 |

The site reactions were acceptable. The serology for Parvo and PRRS (established 52 days post vaccination) were acceptable and deemed to meet the efficacy requirements. The PRRS titre post vaccination was comparable with a titre obtainable with the commercial product Porcilis PRRS, and the parvo HI titre was in line with what is obtainable with the commercial product Porcilis EPL.

In conclusion, the associated non mixed use of the separate safe and effective vaccines EP-ID and L-ID, even when given in conjunction with a live attenuated PRRS vaccine lead to a safe vaccination, effective in protection against and infection with Erysipelothrix rhusiopathiae, porcine parvo virus, various Leptospira bacteria and PRRS virus.

### Example 4

Example 4 is a fourth trial of an Ery-Parvo-Lepto combination vaccine, aiming at establishing the protection against a L-pomona challenge. Basically, the same experimental set-up as in Example 3 was used, except that in this experiment forty 6-week-old piglets were used, divided over four groups. The vaccination of these groups was identical to the vaccination as described in Example 3.

At 11 weeks of age the pigs were transported to a challenge facility and one week later the pigs were challenged intravenously with Leptospira interrogans serogroup Pomona. During 2 weeks after challenge the pigs were observed daily for clinical signs and/or abnormalities in demeanour and appetite. Rectal temperature was measured on regular timepoints until study end which was 2 weeks post-challenge. Serum blood was sampled on day of each vaccination, day of challenge and 2w after challenge and used to determine antibody titres. Heparin blood was sampled just before challenge, at 24 and 28 hours after challenge and on days 2, 3, 4, 7 and 10 after challenge for re-isolation of challenge strain.

At the start of the study, all pigs were seronegative for Leptospira serovar Pomona and PRRS virus. After the vaccinations the group 1 antibody responses to Ery, Parvo and Lepto were equal to or higher compared to group 2, indicating that there is no negative effect of Porcilis PRRS on the take of EP-ID and L-ID.

Vaccination with EP-ID and L-ID induced small transient local reactions at the vaccine administration site (max diameter 4 and 3 cm, respectively). All reactions had disappeared within 2 weeks after vaccination. The vaccinations did not induce any clinical abnormalities.

The post-challenge results are summarized in Table 5 below.

TABLE 5
Post post-challenge data for EP-ID plus L-ID

| Group | Results Lepto challenge: # pos/# total |
|---|---|
| 1 (EP-ID/L-ID/PRRS) | 0/10 |
| 2 (EP-ID/L-ID) | 0/10 |
| 3 (EP-ID 25%/L-ID 25%) | 0/10 |
| 4 (Control) | 9/9 |

The results show that vaccination with EP-ID in associated non-mixed use with L-ID and PRRS, in pigs, is safe. In addition, all three vaccine groups, including associated non-mixed use with the live attenuated PRRS vaccine and the vaccine containing 25% of the normal amount of antigen, induced complete protections against Leptospira interrogans serogroup Pomona.

1. A method for prophylactically treating a pig against an infection with a Leptospira bacterium, comprising injection of a first vaccine comprising a non-replicating Leptospira immunogen into the dermis of the pig.

2. The method of claim 1, the non-replicating Leptospira immunogen is a Leptospira interrogans bacterin.

3. The method of claim 2, wherein the first vaccine additionally comprises a Leptospira kirschneri and/or Leptospira santarosai bacterin.

4. The method of claim 1, wherein the first vaccine comprises an adjuvant.

5. The method of claim 4, wherein the adjuvant is an oil in water adjuvant.

6. The method of claim 1, wherein the first vaccine is injected by a jet stream of this vaccine using a needle-less device, wherein the jet stream penetrates the skin of the pig.

7. The method of claim 1, wherein the first vaccine is used in combination with a second vaccine, the second vaccine comprising a non-replicating immunogen of Erysipelothrix rhusiopathiae and a non-replicating immunogen of porcine parvo virus, wherein the first vaccine and the second vaccine are injected into the dermis of the pig at a first and a second injection site of the pig respectively.

8. The method of claim 7, characterised in that the first and second injection sites are at most 4 cm apart from each other.

9. The method of claim 7, wherein the first vaccine and the second vaccine are injected substantially simultaneously.

10. The method of claim 7, wherein the first and second vaccines are given in a prime and boost vaccination scheme, wherein both the first and second vaccine are injection at the prime vaccination, and both the first and second vaccine are injected at the boost vaccination.

11. The method of claim 7, wherein, the non-replicating immunogen of Erysipelothrix rhusiopathiae is an Erysipelothrix rhusiopathiae bacterin, and the non-replicating immunogen of porcine parvo virus is an inactivated porcine parvo virus.

12. The method of claim 7, further comprising injecting a third vaccine into to the pig, the third vaccine comprising a live attenuated PRRS virus.

13. The method of claim 12, wherein the pig receives a prime vaccination with the first and second vaccine, and a boost vaccination with the first, second and third vaccine wherein the third vaccine is a single dose vaccine.

14. The method of claim 13, wherein the third vaccine is mixed with the first vaccine.

15. A method of preventing infection of a pig with a Leptospira bacterium, comprising administering a first vaccine to the dermis of the pig, wherein the first vaccine comprises a non-replicating Leptospira immunogen.

16. The method of claim 7, wherein the first and second injection sites are at most 3 cm apart from each other.

17. The method of claim 5, wherein the adjuvant does not comprise aluminium hydroxide.

18. The method of claim 17, wherein the adjuvant is X-solve.

19. The method of claim 15, wherein the method comprises a prime vaccination at a first timepoint and a boost vaccination at a second timepoint, wherein
the prime vaccination comprises administration of the first vaccine at a first injection site and the second vaccine at a second injection site, and
the boost vaccination comprises the administration of the first vaccine at the first injection site and the second vaccine at the second injection site.

20. The method of claim 19, wherein the method further comprises preventing an infection in the pig against PRRS, the method further comprising administering a third vaccine comprising a live attenuated PRRS virus, wherein the third vaccine is optionally mixed with the first vaccine.
